(19) United States
(12) Patent Application Publication
(10) Pub. No.: US 2006/0110760 A1
(43) Pub. Date: May 25, 2006

(54) Microarray using laminar flow and method of preparing the same

(76) Inventors: Su-hyeon Kim, Seoul (KR); In-ho Lee, Gyeonggi-do (KR); Jun-hong Min, Gyeonggi-do (KR)

Correspondence Address: CANTOR COLBURN, LLP, 55 GRIFFIN ROAD SOUTH, BLOOMFIELD, CT, 06002, US

(21) Appl. No.: 11/269,037

(22) Filed: Nov. 7, 2005

(30) Foreign Application Priority Data
Nov. 25, 2004 (KR) 10-2004-0097600

(51) Int. Cl. C12Q 1/68; C12M 1/34

(52) U.S. Cl. 435/6; 435/287.2; 977/924

(57) ABSTRACT

A microarray including hydrogel and a plurality of probes which are immobilized in discrete regions of the hydrogel, and a method of preparing the same are provided. When using the microarray and method, a solid substrate is not required and many biomolecules can be immobilized in a small volume, thereby obtaining high sensitivity. Since gel can be cut to obtain many pieces, many microarrays can be prepared at once.

### BACKGROUND OF THE INVENTION

This application claims the benefit of Korean Patent Application No. 10-2004-0097600, filed on Nov. 25, 2004, in the Korean Intellectual Property Office, the disclosure of which is incorporated herein in its entirety by reference.

1. Field of the Invention

The present invention relates to a microarray using laminar flow and a method of preparing the same.

2. Description of the Related Art

Generally, a microarray includes a group of biomolecules, such as polynucleotides or proteins, densely immobilized on a solid substrate. The biomolecules are immobilized within predetermined discrete regions of the substrate. Such microarrays are well known in the art and are described in, for example, U.S. Pat. Nos. 5,445,934 and 5,744,305. Examples of such microarrays include protein and polynucleotide microarrays.

Microarrays are generally manufactured using photolithography. When photolithography is used, a polynucleotide array can be manufactured by repeatedly exposing to an energy source a predetermined discrete region of a substrate on which a monomer protected by a removable group is coated to remove the protecting group, and coupling the deprotected monomer with another monomer protected by the removable group. Alternatively, pre-synthesized polynucleotides can be immobilized in predetermined discrete regions of a substrate. Immobilization methods, which are used only in this case, include a spotting method, a piezoelectric printing method using inkjet printer, and a micro pipetting method. The method of immobilizing already synthesized biomolecules on a substrate is widely used since it can be used to array biomolecules in various patterns.

However, in the methods of preparing a microarray as described above, probes, which are molecules immobilized on the microarray that specifically bind to target molecules, are sequentially immobilized on a substrate. Thus, the time required for immobilization is proportional to the types of probes and the number of microarrays and it is difficult to adjust the amount of a probe on the substrate exactly. In addition, the material composing the solid substrate, such as glass, silicone, etc., is limited when the chip size is reduced.

U.S. Patent Application Publication No. 20030124509 discloses a method of forming a micropattern using laminar flow, but does not describe a microarray and photopolymerization.

U.S. Patent Application Publication No. 20030116437 discloses electrophoresis in microfabricated devices using photopolymerized polyacrylamide gels. However, the aim of the invention is to use polyacrylamide gels in electrophoresis and there is no description regarding the preparation of a microarray.

Thus, the aim of the present invention is to overcome the above problems with a microarray having a gel form, which does not require a solid substrate, and can be prepared by one-dimensionally arranging probes using laminar flow and immobilizing the probes using photopolymerization.

### SUMMARY OF THE INVENTION

The present invention provides a microarray using laminar flow, which does not require a solid substrate and contains many probes immobilized in a small area to obtain high sensitivity, and a method of preparing the same.

According to an aspect of the present invention, there is provided a microarray including hydrogel and a plurality of probes which are immobilized in discrete regions of the hydrogel.

In the microarray, the hydrogel may be prepared by polymerizing a monomer having an ethylene group. The monomer may be selected from the group consisting of acrylamide, methacrylamide, acrylic acid, methacrylic acid, and amides and esters having structures similar to the structures of said compounds.

In the microarray, the probes may be covalently bound to the hydrogel and immobilized in the hydrogel directly or using spacers. The spacers may be microparticles or nanoparticles.

In the microarray, the microparticles or nanoparticles may be immobilized in the hydrogel by covalent bonds or by embedding and include microbeads, nanobeads, colloidal particles, bioparticles, etc.

In the microarray, the probes may be biomolecules. The biomolecules may be selected from the group consisting of DNA, RNA, peptide nucleic acid (PNA), locked nucleic acid (LNA), protein, and cells.

According to another aspect of the present invention, there is provided a method of preparing a microarray using an apparatus including a plurality of channels and an integration channel connected to the plurality of channels, the method including: introducing a mixture of a photopolymerizable compound-containing solution and probes into the integration channel via the plurality of channels such that the probes from the channels have laminar flow; photopolymerizing the solution by irradiating radiation onto the integration channel to produce hydrogel; and separating the hydrogel from the channel.

In the method, the irradiating radiation onto the integration channel may be performed through a photomask to photopolymerize part of the solution.

The method may further include separating the photopolymerized hydrogel from the mixture.

The method may further include cutting the separated hydrogel.

In the method, the laminar flow of the probes may be induced by sucking the probes from the integration channel using a pump.

In the method, the photopolymerizable compound may be a monomer having an ethylene group. The compound may be selected from the group consisting of acrylamide, methacrylamide, acrylic acid, methacrylic acid, and amides and esters having structures similar to the structures of said compounds.

In the method, the probes may be immobilized on microparticles or nanoparticles. The microparticles or nanoparticles may include microbeads, nanobeads, colloidal particles, bioparticles, etc.

In the method, the probes may be biomolecules. The biomolecule may be selected from the group consisting of DNA, RNA, PNA, LNA, protein, and cells.

According to another aspect of the present invention, there is provided a laminar flow generating apparatus for the preparation of a hydrogel microarray, including a plurality of channels and an integration channel connected to the plurality of channels.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic diagram illustrating an embodiment of a method of forming laminar flow in order to prepare a microarray of the present invention;

FIG. 2 is a schematic diagram of hydrogel produced by photopolymerization;

FIG. 3 is a schematic diagram of one-dimensional microarrays in the forms of bars, obtained by cutting the hydrogel;

FIG. 4 is a schematic diagram of a laminar flow generating apparatus for preparation of a hydrogel microarray according to an embodiment of the present invention;

FIG. 5 shows laminar flow formed by a capillary array in Example 1;

FIG. 6 is a microscopic photograph of a photopolymerized hydrogel;

FIG. 7A is a schematic diagram illustrating relative positions of channels on a chip; and

FIG. 7B is a microscopic photograph of fluid at a point where 8 of the channels of FIG. 7A integrate.

### DETAILED DESCRIPTION OF THE INVENTION

Hereinafter, the present invention will be described in more detail.

The present invention relates to a microarray including hydrogel and a plurality of probes which are immobilized in discrete regions of the hydrogel.

In a conventional microarray, probes are immobilized on a solid substrate. However, in the present invention, probes are immobilized in hydrogel without using a solid substrate. Hydrogel refers to a gel containing water and can be used to immobilize a plurality of probes. The hydrogel can be cut to form a one-dimensional microarray. Since the hydrogel can be easily cut, it is suitable for the present invention. Further, hydrophilic biomolecules, such as nucleic acids, etc., can be easily penetrated into hydrogel, and thus the reaction rate between the hydrophilic biomolecule and hydrogel is high.

A plurality of probes are introduced through separate channels and integrated in an integration channel. In the integration channel, laminar flow is induced so that the probes remain separated. When ultraviolet (UV) rays are irradiated onto the integration channel to induce photopolymerization, layers of the probes are immobilized in separate states in the hydrogel. Thus, the respective probes are immobilized in discrete regions of the hydrogel.

In an embodiment of the present invention, the hydrogel may be prepared by polymerizing monomer having an ethylene group. The monomer is a polymerizable compound, such as acrylamide, methacrylamide, acrylic acid, methacrylic acid, an amide or ester having a structure similar to the structures of said compounds, or the like. A polyacrylamide gel may be used as the hydrogel.

In an embodiment of the present invention, the probes may covalently bind to the hydrogel. The probes may be immobilized in the hydrogel by covalent bond caused by copolymerization. Any method that allows the probes to covalently bind to the hydrogel may be used in the present invention.

In an embodiment of the present invention, the probes can be immobilized in the hydrogel through spacers. The spacers can be microparticles or nanoparticles. When the probes flow in the integration channel, they may diffuse. To reduce the diffusion, the probes can be immobilized in the hydrogel by spacers, such as microparticles or nanoparticles. The nanoparticles have a greater diameter than pores of the hydrogel, and thus cannot be emitted from the hydrogel. The pore size of the hydrogel varies according to a concentration of a gel and a degree of polymerization, but an average diameter can be several nanometers. Therefore, the nanoparticles should have a diameter greater than several nanometers so as not to be emitted from the hydrogel. In the present invention, the diameter of the nanoparticles can be several nm to 100 nm. The probes may be immobilized by nanoparticles using a variety of methods. For example, streptavidin can be fixed to the nanoparticle surface and biotin bound to the terminal of nucleic acid, and then the nucleic acid may be immobilized on the nanoparticle through a strong bond between the streptavidin and biotin. When the nanoparticles are composed of or coated with metals that can bind to a thiol group, such as gold etc., a thiol group attaches to the nucleic acid, thereby immobilizing the nucleic acid on the nanoparticle through a covalent bond between the metal and the thiol group. When silica particles are used, nucleic acids may be immobilized on the silica particle using silane. These methods of immobilizing nucleic acids on the nanoparticles are well-known to those skilled in the field of surface synthesis. The nucleic acids may be immobilized in a larger surface area when using nanoparticles than when nucleic acids are immobilized on a flat surface. Thus, more nucleic acids can be immobilized.

In an embodiment of the present invention, the microparticles or nanoparticles may be immobilized in the hydrogel by covalent bonds or by embedding. Embedding refers to a procedure of preparing a sample that has been penetrated appropriately to be sliced using a microtome. Recently, in most laboratories, an embedding center for automatically embedding has been used.

In an embodiment of the present invention, the microparticles or nanoparticles may be micro beads, nano beads, colloidal particles, bioparticles, etc. Any particle, which can bind to the probe and hydrogel and does not cause diffusion in the integration channel may be used.

In an embodiment of the present invention, the probes may be biomolecules. The biomolecules may be selected from the group consisting of DNA, RNA, peptide nucleic acid (PNA), locked nucleic acid (LNA), protein, and cells.

The present invention also relates to a method of preparing a microarray using an apparatus including a plurality of channels and an integration channel connected to the plurality of channels, the method including: introducing a mixture of a photopolymerizable compound-containing solution and probes into the integration channel via the plurality of channels such that the probes from the channels have laminar flow; photopolymerizing the solution by irradiating radiation onto the integration channel to produce hydrogel; and separating the hydrogel from the channel.

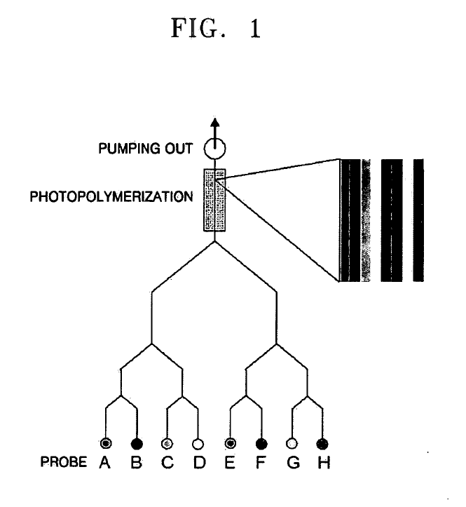

In the method of preparing a microarray of the present invention, hydrogel is used, not a solid substrate. In the method, laminar flow is formed to separate the respective probe layers and radiation is irradiated to photopolymerize the photopolymerizable compound, thereby forming the hydrogel. FIG. 1 is a schematic diagram illustrating the formation of laminar flow in order to prepare a microarray according to an embodiment of the present invention. Referring to FIG. 1, a plurality of probes (in FIG. 1, 8 probes) are introduced into a plurality of channels, respectively. The probes introduced into the respective channels form laminar flow in an integration channel. Laminar flow is the flow of fluid with a constant velocity at each point. For example, if water flows in a narrow pipe and its flow state is observed using ink, the ink flows linearly when the Reynolds number is small, indicating that the water runs parallel to the pipe wall.

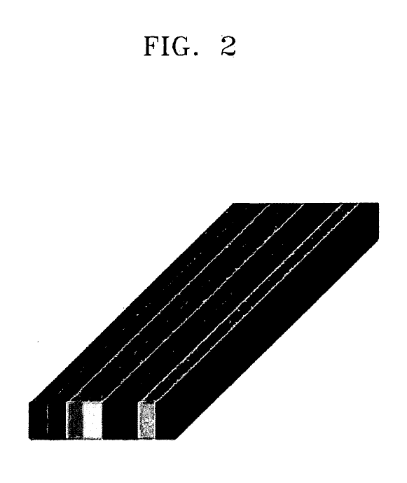

When probe layers are formed by the laminar flow, radiation is irradiated to photopolymerize the photopolymerizable compound, thereby forming the hydrogel. FIG. 2 is a schematic diagram of hydrogel produced using photopolymerization. Referring to FIG. 2, a hydrogel having 8 probes of different colors immobilized therein is produced. Photopolymerization is caused by the irradiation of radiation and is classified into pure photopolymerization and photosensitive polymerization. Both methods require UV rays or visual rays. In pure polymerization, when radiation is irradiated onto a compound (monomer) having a relatively low molecular weight, which is a basic repeating unit in a polymer structure, the compound (monomer) absorbs the radiation and is activated, resulting in polymerization. For example, when UV rays are irradiated onto methyl acrylate, polymethylacrylate is obtained. In photosensitive polymerization, when a small quantity of another material (photosensitizer) is added to a compound to be polymerized and radiation is irradiated thereon, the other material absorbs light and is activated, thereby causing polymerization. For example, when 5-nitrofluorene as a photosensitizer is added to a cinnamic ester of polyvinylalcohol and radiation is irradiated thereon, a resin insoluble in a solvent can be obtained by crosslinking.

In an embodiment of the present invention, the irradiating radiation onto the integration channel is performed through a photomask to photopolymerize a part of the solution. The photomask can be used to irradiate radiation to only a region to be photopolymerized, thereby forming alternate photopolymerized regions and non-photopolymerized regions in the integration channel. Thus, the obtained hydrogel need not be cut. Alternatively, the whole solution containing the photopolymerizable compound may be photopolymerized by irradiating radiation onto the whole integration channel.

In an embodiment of the present invention, the method of preparing a microarray may further include separating the photopolymerized hydrogel from the mixture. Since the hydrogel obtained using the photomask has photopolymerized regions and non-photopolymerized regions, an operation of separating the photopolymerized regions is required. The separated hydrogel may be a one-dimensional microarray in the form of a bar.

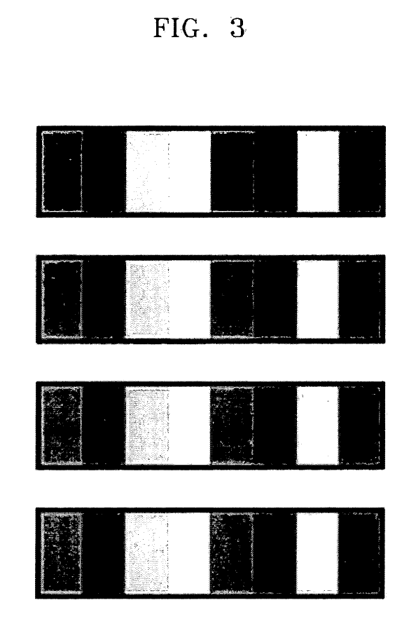

In an embodiment of the present invention, the method of preparing a microarray may further include cutting the separated hydrogel. A hydrogel produced without photomasking should be cut to appropriate sizes. FIG. 3 is a schematic diagram of one-dimensional microarrays in the form of bars, obtained by cutting the resulting hydrogel. The microarrays in the bar forms may be obtained by using a tool or instrument capable of cutting the hydrogel into pieces with widths of 5 mm or less. Any tool or instrument capable of cutting the hydrogel, for example, a knife, microtome, or the like, may be used in the present invention.

The one-dimensional microarrays are placed in a container, such as an eppendorf tube or a 96-well plate, and reacted with the target sample, and then detection is performed using a fluorescent measurement, or the like after washing.

In an embodiment of the present invention, the flowing may be performed by sucking the probes from the integration channel using a pump. To produce laminar flow, probes may be injected by pumping using the respective pumps in a plurality of channels. However, this method is not preferable since as many pumps as probes are required. Thus, it is preferable to suck the probes from the integration channel, since only one pump is needed, regardless of the number of probes. That is, pumping out is preferable.

In an embodiment of the present invention, the hydrogel may be prepared by polymerizing a monomer having an ethylene group. The monomer is a polymerizable compound which includes acrylamide, methacrylamide, acrylic acid, methacrylic acid, or an amide or ester having a structure similar to the structures of said compounds, etc. A polyacrylamide gel may be used as the hydrogel.

In an embodiment of the present invention, the probes can be immobilized on microparticles or nanoparticles. When the probes flow in the integration channel, diffusion thereof may occur. To reduce the diffusion, in an embodiment of the present invention, the probes can be bound to microparicles or nanoparticles. The probes may be immobilized by nanoparticles using a variety of methods. For example, streptavidin can be fixed to the nanoparticle surface and biotin bound to the terminal of nucleic acid, and then the nucleic acid may be immobilized on the nanoparticle surface through a strong bond between the streptavidin and biotin. When the nanoparticles are composed of or coated with metals that can bind to a thiol group, such as gold etc., a thiol group attaches to the nucleic acid, thereby immobilizing the nucleic acid on the nanoparticle through a covalent bond between the metal and the thiol group. When the nanoparticles are silica particles, nucleic acids may be immobilized on the silica particle using silane chemistry. These methods of immobilizing nucleic acids on the nanoparticles are well-known to those skilled in the field of surface synthesis. The nucleic acids may be immobilized in a larger surface area when using nanoparticles than when nucleic acids are immobilized on a flat surface. Thus, more nucleic acids can be immobilized.

In an embodiment of the present invention, the microparticles or nanoparticles may be micro beads, nano beads, colloidal particles, bioparticles, etc. Any particle which can bind to the probe and hydrogel and does not cause diffusion in the integration channel may be used.

In an embodiment of the present invention, the probes may be biomolecules. The biomolecules may be selected from the group consisting of DNA, RNA, PNA, LNA, protein, and cell.

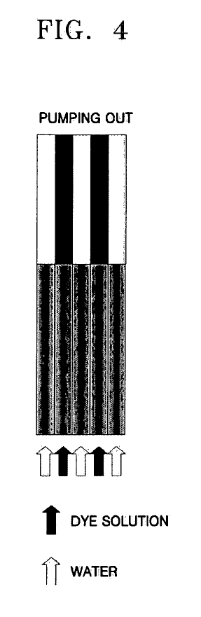

The present invention also relates to a laminar flow generating apparatus for the preparation of a hydrogel microarray, including a plurality of channels and an integration channel connected to the plurality channels. FIG. 4 is a schematic diagram of a laminar flow generating apparatus for the preparation of a hydrogel microarray according to an embodiment of the present invention. Referring to FIG. 4, the apparatus includes 5 channels alternately filled with a dye solution and water. When the solution is sucked from the top of the apparatus by a pump, all fluids that flow through the respective channels are injected into an integration channel, which is disposed at terminals of the channels. The fluids that pass through the 5 channels flow into the integration channel while maintaining laminar flow. In FIG. 4, dye solutions (2nd and 4th capillaries) emitted from dye solution channels and water (1st, 3rd, and 5th capillaries) emitted from water channels maintain their flow paths and are not mixed with each other. This phenomenon is possible only in the case of laminar flow and mixing occurs in the case of turbulent flow.

The present invention will now be described in greater detail with reference to the following examples. The following examples are for illustrative purposes only and are not intended to limit the scope of the invention.

### EXAMPLES

### Example 1

### Formation of Laminar Flow by Capillary Array

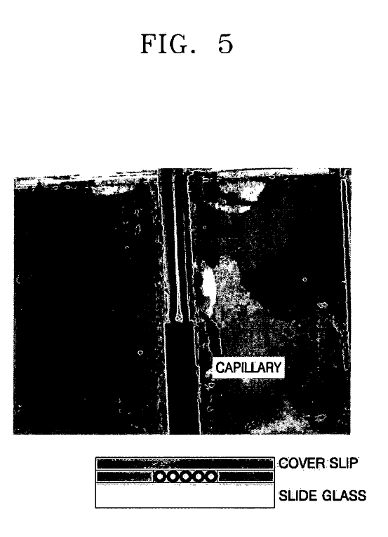

As illustrated in FIG. 4, 5 channels were made in one end of a capillary array and an integration channel was made in the other end of the capillary array. A phenolphthalein dye solution and water, respectively, were allowed to flow through the capillary array and the formation of laminar flow was investigated. FIG. 5 illustrates laminar flow that was formed by the capillary array. In the experiment, 5 capillaries were placed on a slide glass and a cover slip was placed thereon. Then, the dye solution (2nd and 4th capillaries) and water (1st, 3rd, and 5th capillaries) were allowed to flow through the capillaries. As a result, 2 dye solution bands resulting from the dye solution that was passed through 2nd and 4th capillaries were observed, which indicated laminar flow in the integration channel. Thus, it can be seen that when a solution containing different probes and a photopolymerizable monomer are introduced instead of the dye solution and water into a plurality of channels, the probes can be separated into discrete regions in an integration channel. UV rays were irradiated onto the integration channel to obtain a microarray in which the respective probes were immobilized in discrete regions.

### Example 2

Photopolymerization by UV Irradiation

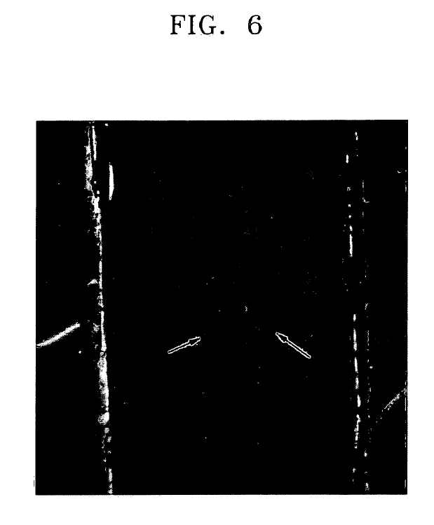

To investigate whether layers of probes separated by laminar flow were immobilized by photopolymerization, photopolymerizable Reprogel™ was used instead of water and a mixture of Reprogel™ (available from Amersham) and Dynabeads® M-270 (available from Dynal Biotech) with a diameter of 2.8 μm was used instead of the dye solution to form laminar flow. Then, the laminar flow was stopped while irradiating UV rays with a wavelength of 302 nm to carry out photopolymerization. FIG. 6 is a microscopic photograph of the photopolymerized hydrogel. Referring to FIG. 6, two distinct bead bands indicated by arrows immobilized in the channel by photopolymerization were observed. This indicates that a microarray in which layers of the probes separated by laminar flow are immobilized can be prepared.

### Example 3

### Formation of Laminar Flow in a Plurality of Channels on a Chip

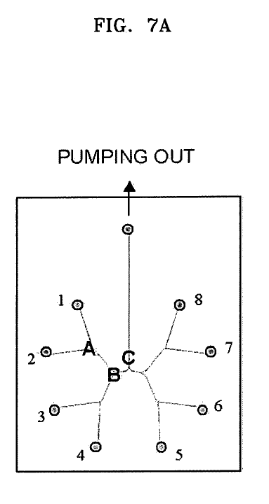

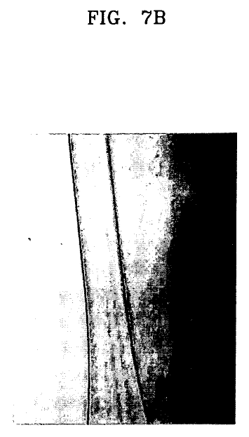

Dynabeadsφ were injected into a plurality of channels on a chip and the solution was sucked with a syringe pump to observe the formation of laminar flow. FIG. 7A schematically illustrates relative positions of the respective channels and FIG. 7B is a microscopic photograph of fluid at a point C where 8 channels are integrated. Referring to FIG. 7A, the beads were introduced into channels 2, 4, 6, and 8 on the chip. Referring to FIG. 7B, the beads introduced into channels 2, 4, 6, and 8 were observed in the integration channel as four distinct bands. Thus, when probes are introduced into a plurality of channels which are spatially separated on a chip, a microarray of the present invention can be prepared.

As described above, according to the present invention, laminar flow is used to form a pattern of layers arranged in parallel and the pattern is immobilized by photopolymerization to obtain an array. In this way, a microarray of DNA, protein, etc. can be prepared by immobilizing biomolecules in the form of beads. Moreover, a solid substrate is not required and many biomolecules can be immobilized in a small area, thereby obtaining high sensitivity. Since gel can be cut to obtain many pieces, many microarrays can be prepared at once.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A microarray comprising hydrogel and a plurality of probes which are immobilized in discrete regions of the hydrogel.

2. The microarray of claim 1, wherein the hydrogel is prepared by polymerizing a monomer having an ethylene group.

3. The microarray of claim 2, wherein the monomer is selected from the group consisting of acrylamide, methacrylamide, acrylic acid, methacrylic acid, and amides and esters having structures similar to the structures of said compounds.

4. The microarray of claim 1, wherein the probes are covalently bound to the hydrogel.

5. The microarray of claim 1, wherein the probes are immobilized in the hydrogel using spacers.

6. The microarray of claim 5, wherein the spacers are microparticles or nanoparticles.

7. The microarray of claim 6, wherein the microparticles or nanoparticles are immobilized in the hydrogel by covalent bonds or by embedding.

8. The microarray of claim 6, wherein the microparticles or nanoparticles are selected from the group consisting of microbeads, nanobeads, colloidal particles, and bioparticles.

9. The microarray of claim 1, wherein the probes are biomolecules.

10. The microarray of claim 9, wherein the biomolecules are selected from the group consisting of DNA, RNA, PNA, LNA, protein, and cells.

11. A method of preparing a microarray using an apparatus comprising a plurality of channels and an integration channel connected to the plurality of channels, the method comprising:
introducing a mixture of a photopolymerizable compound-containing solution and probes into the integration channel via the plurality of channels such that the probes from the channels have laminar flow;
photopolymerizing the solution by irradiating radiation onto the integration channel to produce hydrogel; and
separating the hydrogel from the channel.

12. The method of claim 11, wherein the irradiating radiation onto the integration channel is performed through a photomask to photopolymerize part of the solution.

13. The method of claim 12, further comprising separating the photopolymerized hydrogel from the mixture.

14. The method of claim 11, further comprising cutting the separated hydrogel.

15. The method of claim 11, wherein the laminar flow of the probes is induced by sucking the probes from the integration channel through a pump.

16. The method of claim 11, wherein the photopolymerizable compound is a monomer having an ethylene group.

17. The method of claim 16, wherein the compound is selected from the group consisting of acrylamide, methacrylamide, acrylic acid, methacrylic acid, and amides and esters having structures similar to the structures of said compounds.

18. The method of claim 11, wherein the probes are immobilized on microparticels or nanoparticles.

19. The method of claim 18, wherein the microparticles or nanoparticles are selected from the group consisting of microbeads, nanobeads, colloidal particles, and bioparticles.

20. The method of claim 11, wherein the probes are biomolecules.

21. The method of claim 20, wherein the biomolecules are selected from the group consisting of DNA, RNA, PNA, LNA, protein, and cells.

22. A laminar flow generating apparatus for the preparation of a hydrogel microarray, comprising a plurality of channels and an integration channel connected to the plurality of channels.
